(19) United States
(12) Patent Application Publication
(10) Pub. No.: US 2024/0148513 A1
(43) Pub. Date: May 9, 2024

(54) MODULAR ARTIFICIAL FINGER JOINT IMPLANT

(71) Applicants: CHIH-HAO CHANG, TAIPEI (TW); HUANG LIANG BIOMEDICAL TECHNOLOGY CO., LTD., Kaohsiung City (TW)

(72) Inventor: CHUNG CHUN HSUEH, KAOHSIUNG CITY (TW)

(21) Appl. No.: 17/981,942

(22) Filed: Nov. 7, 2022

(51) Int. Cl. A61F 2/42

(52) U.S. Cl. CPC A61F 2/4241; A61F 2002/4243

(57) ABSTRACT

A modular artificial finger joint implant has a first finger joint module and a second finger joint module. The first finger joint module includes a first connecting structure connecting a first joint head element and a first joint root element. The second finger joint module includes a second connecting structure connecting a second joint head element and a second joint root element. A first articular facet of the first joint head element and a second articular facet of the second joint head element are slidable relative to each other. Since the modular artificial finger joint implant is modular in structure and has a variety of changeable combinations, manufacturers' costs on manufacturing and inventories can be reduced. Moreover, a surgery for replacing damaged parts can be performed, so as to reduce cost on replacing.

### BACKGROUND OF THE INVENTION

### 1. Field of the Invention

The present invention relates to an artificial finger joint implant for an affected digit (fingers or toes) of a human body, especially to a modular artificial finger joint implant.

### 2. Description of the Prior Art(s)

Regarding injuries to cartilages of a metacarpophalangeal joint and an interphalangeal joint, and for patients suffering from ankyloses which surgical release is ineffective or with joint functions that cannot be restored, the use of artificial digit joint replacement surgery to preserve the joint functions is currently the most useful treatment method.

A conventional artificial digit joint implant includes a first digit joint element and a second digit joint element. Each of the first digit joint element and the second digit joint element is an integrally formed single part and has an upper portion (head portion) and a lower portion (root portion). The first digit joint element and the second digit joint element are embedded into two phalanges with the lower portions (root portions) respectively. Then the upper portions (head portions) of the first digit joint element and the second digit joint element are connected with each other through matching prosthetic surfaces and are slidable relative to each other. Accordingly, the conventional artificial digit joint implant is able to work like a joint.

However, internationally, regarding sizes of the artificial digit joint implant, on the basis of the upper portion (head portion) and the lower portion (root portion), each of the first digit joint element and the second digit joint element has 7-8 different sizes that can be chosen from. Since the upper portion (head portion) and the lower portion (root portion) are integrally formed as a single part, i.e. the first (second) digit joint elements with one size of the upper portion may have 7-8 different sizes of the lower portions, manufacturers have to ship the conventional artificial digit joint implants according to the sizes of the upper portion (head portion) and the lower portion (root portion) that are required by the doctors. The upper portion (head portion) and the lower portion (root portion) of one first (second) digit joint element are unchangeable since the upper portion (head portion) and the lower portion (root portion) are integrally formed as a single part. Consequently, the manufacturers is under the pressure to accumulated inventories.

In addition, since each of the first digit joint element and the second digit joint element is an integrally formed single part, when the first digit joint element and the second digit joint element are implanted to an affected digit of a human body and any one of the upper portions (head portion) of the first digit joint element and the second digit joint element wears out, a surgery to replace the first digit joint element or the second digit joint element has to be implemented, which is time-consuming and cost a lot.

To overcome the shortcomings, the present invention provides a modular artificial finger joint implant to mitigate or obviate the aforementioned problems.

### SUMMARY OF THE INVENTION

The main objective of the present invention is to provide a modular artificial finger joint implant that has a first finger joint module and a second finger joint module.

The first finger joint module includes a first joint head element having a first articular facet, a first joint root element, and a first connecting structure. The first connecting structure is disposed between and connects the first joint head element and the first joint root element, and includes a first connecting portion and a first combining portion. The first connecting portion and the first combining portion are detachably connected with each other.

The second finger joint module includes a second joint head element having a second articular facet, a second joint root element, and a second connecting structure. The second articular facet is slidable relative to the first articular facet of the first joint head element. The second connecting structure is disposed between and connects the second joint head element and the second joint root element, and includes a second connecting portion and a second combining portion. The second connecting portion and the second combining portion are detachably connected with each other.

The modular artificial finger joint implant has the following advantages.

First, the modular artificial finger joint implant of the present invention has a variety of changeable combinations, which can reduce manufacturers' costs on manufacturing and inventories. Since the modular artificial finger joint implant is modular in structure, with the first/second connecting portion and the first/second combining portion of the first/second connecting structure between the first/second joint head element and the first/second joint root element, the first/second joint head elements of different types and the first/second joint root element of different types can be combined according to users' needs. Therefore, the modular artificial finger joint implant of the present invention has a variety of changeable combinations. Accordingly, it is able to quickly select appropriate types of the first/second joint head elements and the first/second joint root elements during surgery. Furthermore, the manufacturers' costs on manufacturing and inventories can be reduced.

Second, the modular artificial finger joint implant of the present invention is replaceable. As described above, the modular artificial finger joint implant is modular in structure. With the first/second connecting portion and the first/second combining portion of the first/second connecting structure between the first/second joint head element and the first/second joint root element, when the modular artificial finger joint implant has been implanted to a finger of a toe of a human body, a surgery for replacing damaged parts can be performed, so as to reduce cost on replacing the modular artificial finger joint implant.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of a modular artificial finger joint implant in accordance with the present invention;

FIG. 2 is an exploded perspective view of the modular artificial finger joint implant in FIG. 1;

FIG. 3 is a further exploded perspective view of the modular artificial finger joint implant in FIGS. 1 and 2;

FIG. 4 is an exploded perspective view of a first finger joint module of the modular artificial finger joint implant in FIGS. 1 to 3;

FIG. 5 is another exploded perspective view of the first finger joint module of the modular artificial finger joint implant in FIGS. 1 to 3;

FIG. 6 is an exploded perspective view of a second finger joint module of the modular artificial finger joint implant in FIGS. 1 to 3;

FIG. 7 is another exploded perspective view of the second finger joint module of the modular artificial finger joint implant in FIGS. 1 to 3; and

FIG. 8 is an operational side view of the modular artificial finger joint implant in FIG. 1.

### DETAILED DESCRIPTION OF THE PREFERRED EMBODIMENTS

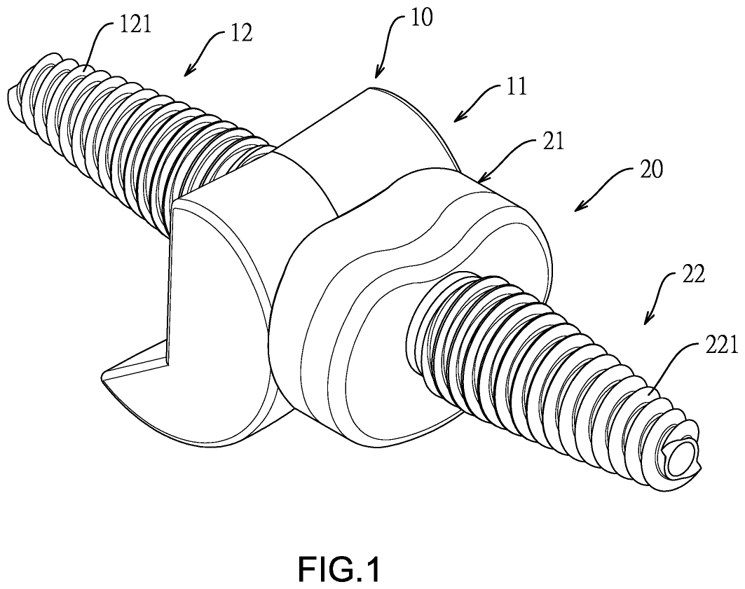

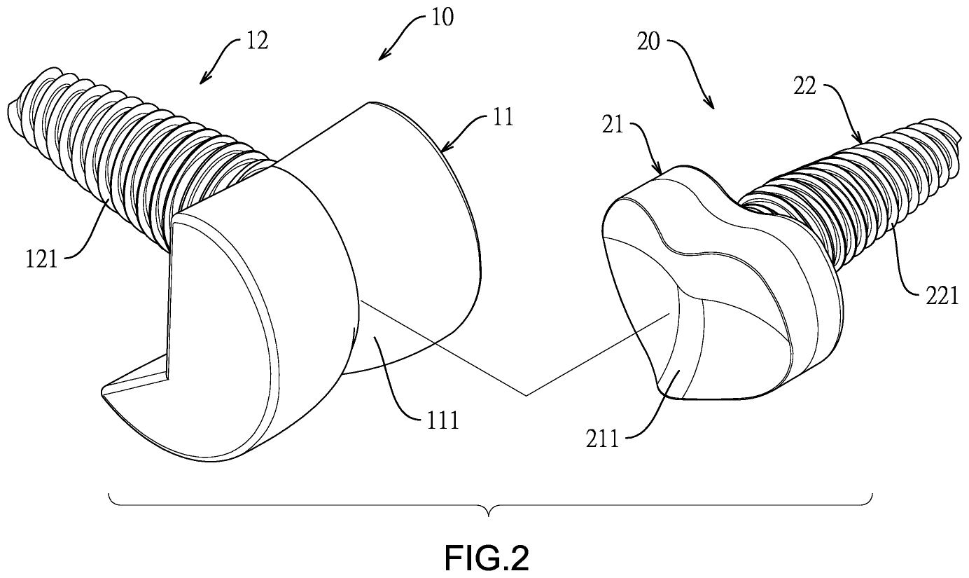

With reference to FIGS. 1 and 2, a modular artificial finger joint implant in accordance with the present invention is shown. In addition to be used to replace a finger joint of a human body, the modular artificial digit finger joint implant may also be used to replace a toe joint of a human body. The modular artificial finger joint implant comprises a first finger joint module 10 and a second finger joint module 20.

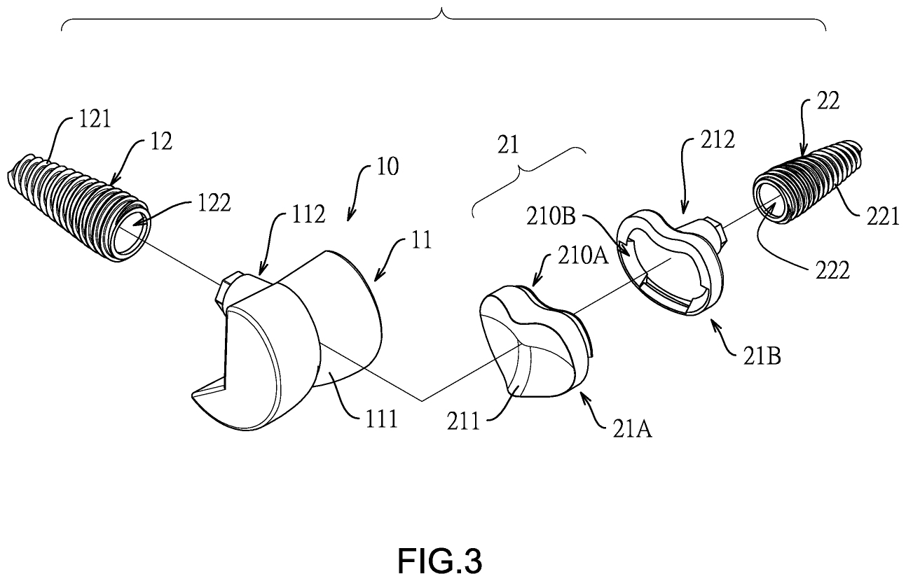

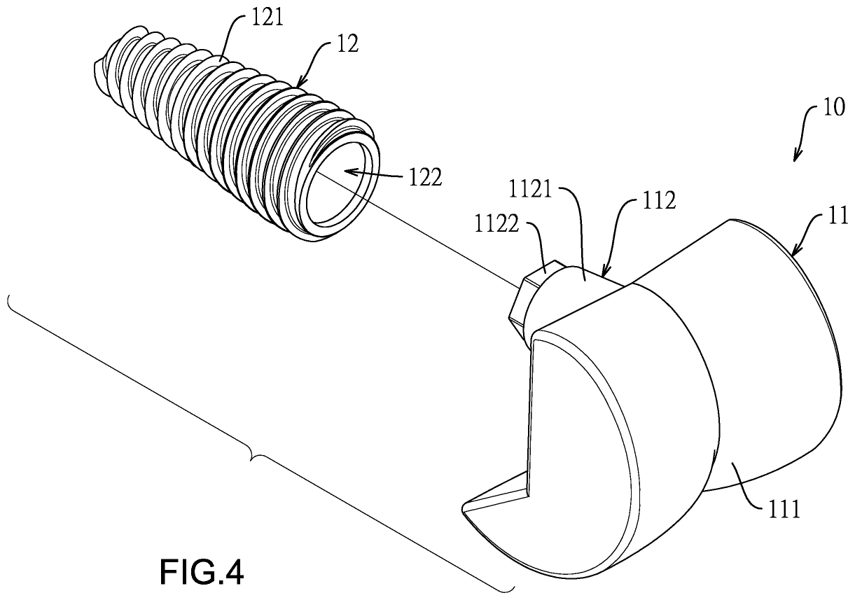

With further reference to FIGS. 3 and 4, the first finger joint module 10 includes a first joint head element 11, a first joint root element 12, and a first connecting structure. The first joint head element 11 has a first articular facet.

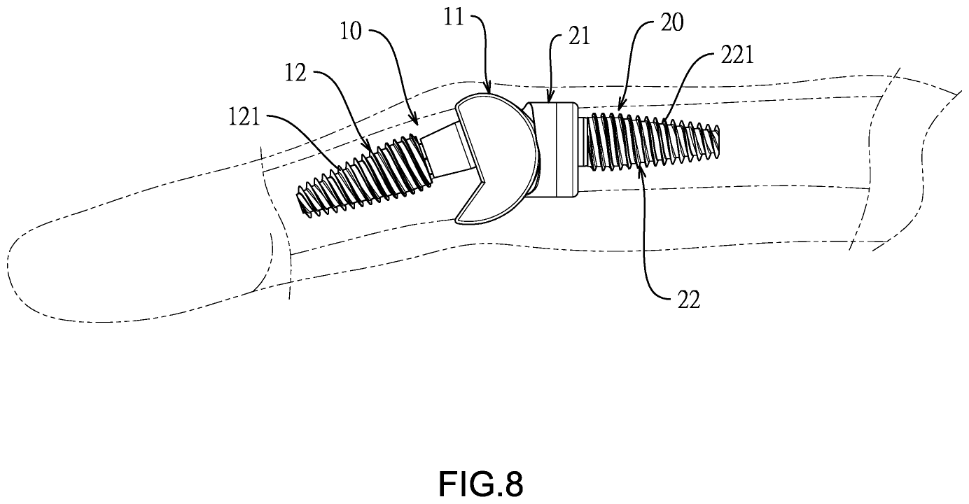

With further reference to FIGS. 3, 4 and 8, the first joint root element 12 is attached to the first joint head element 11, is conical-shaped, and has a thread 121 formed on an outer side surface of the first joint root element 12, such that the first joint root element 12 is able to be screwed into a phalange. With the conical shape, pressure between the first joint root element 12 and said phalange is lowered. Moreover, the thread 121 of the first joint root element 12 forms a tight bond between the first joint root element 12 and a bone tissue while being screwed into said phalange, so as to provide a solid foundation to subsequent combinations of the modular artificial finger joint implant and said phalanges.

Preferably, the outer side surface, on which the thread 121 of the first joint root element 12 is formed, may be formed as a rough surface through a surface finishing process such as sandblasting. With further acid etching, a plurality of sub-micron holes are formed on the outer side surface, on which the thread 121 of the first joint root element 12 is formed. Accordingly, the outer surface of the first joint root element 12 is suitable for attachment of bone cells, which is beneficial for integration of the phalange and the first finger joint module 10.

The first connecting structure is disposed between and connects the first joint head element 11 and the first joint root element 12. Specifically, the first connecting structure includes a first connecting portion 112 and a first combining portion 122. The first joint head element 11 and the first joint root element 12 are detachably connected with each other through the first connecting portion 112 and the first combining portion 122 that match with one another.

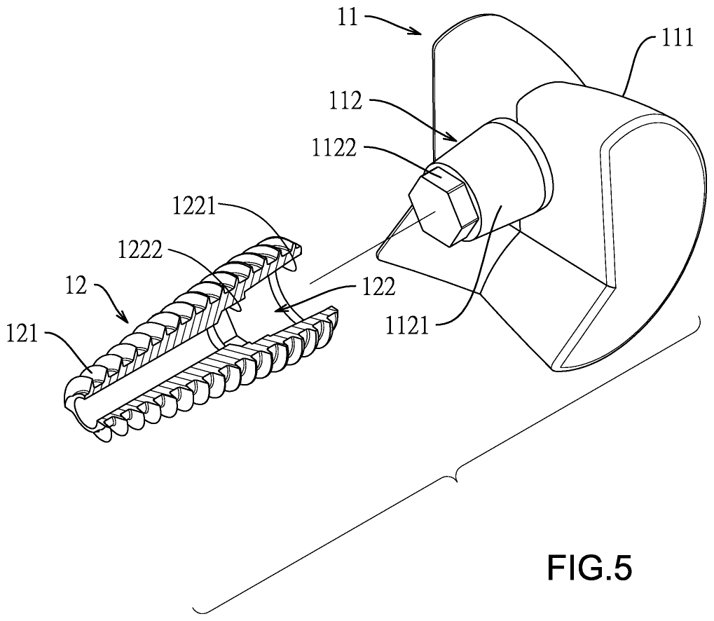

Specifically, with further reference to FIGS. 4 and 5, the first joint head element 11 has a first joint head portion 111. The first articular facet is formed on an end of the first joint head portion 111. The first connecting portion 112 is formed at another end of the first joint head portion 111. The first combining portion 122 is formed at an end of the first joint root element 12. The first connecting portion 112 and the first combining portion 122 is concave-convex matching structure.

Specifically, the first combining portion 122 has a conical recess 1221 and a polygonal recess 1222. The conical recess 1221 of the first combining portion 122 tapers off toward an inner bottom that is defined in said conical recess 1221. The polygonal recess 1222 of the first combining portion 122 is formed in the inner bottom that is defined in the conical recess 1221. The first connecting portion 112 has a conical protrusion 1121 and a polygonal protrusion 1122. The conical protrusion 1121 of the first connecting portion 112 tapers off from a proximal end of the conical protrusion 1121 to a distal end of the conical protrusion 1121. The polygonal protrusion 1122 of the first connecting portion 112 is formed on the distal end of the conical protrusion 1121 of the first connecting portion 112. The conical protrusion 1121 of the first connecting portion 112 is capable of being fitted in the conical recess 1221 of the first combining portion 122. The conical protrusion 1121 and the conical recess 1221 of the first combining portion 122 may be circular or polygonal in cross-section. The polygonal protrusion 1122 of the first connecting portion 112 is capable of being fitted in the polygonal recess 1222 of the first combining portion 122. With the mutually fitted conical protrusion and recess and the mutually fitted polygonal protrusion and recess, the first connecting portion 112 and the first combining portion 122 are able to be connected with each other quickly and stably.

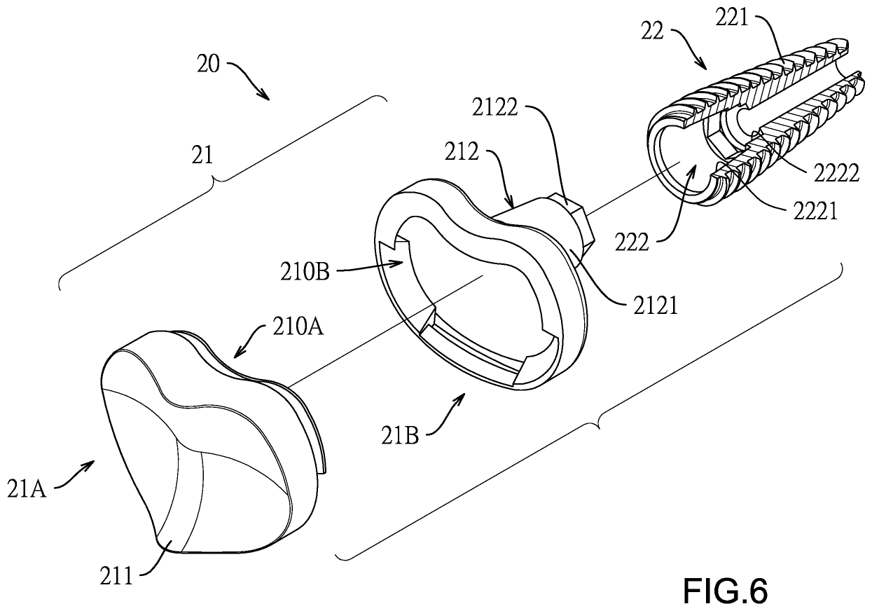

With further reference to FIG. 6, the second finger joint module 20 includes a second joint head element 21, a second joint root element 12, and a second connecting structure. The second joint head element 21 has a second articular facet. The second articular facet matches with and is slidable relative to the first articular facet of the first joint head element 11. Moreover, the first articular facet and the second articular facet are formed as unrestricted bionic surfaces.

With further reference to FIGS. 3 and 6 to 8, the second joint root element 22 is attached to the second joint head element 21, is conical-shaped, and may have a thread 221 formed on an outer side surface of the second joint root element 22, such that the second joint root element 22 is able to be screwed into a phalange. With the conical shape, pressure between the second joint root element 22 and said phalange is lowered. Moreover, the thread 221 of the second joint root element 22 forms a tight bond between the second joint root element 22 and a bone tissue while being screwed into said phalange, so as to provide a solid foundation to subsequent combinations of the modular artificial finger joint implant and said phalanges.

Preferably, the outer side surface, on which the thread 221 of the second joint root element 22 is formed, may be formed as a rough surface through a surface finishing process such as sandblasting. With further acid etching, a plurality of sub-micron holes are formed on the outer side surface, on which the thread 221 of the second joint root element 22 is formed. Accordingly, the outer surface of the second joint root element 22 is suitable for attachment of bone cells, which is beneficial for integration of the phalange and the second finger joint module 20.

The second connecting structure is disposed between and connects the second joint head element 21 and the second joint root element 22. Specifically, the second connecting structure includes a second connecting portion 212 and a second combining portion 222. The second joint head element 21 and the second joint root element 22 are detachably connected with each other through the second connecting portion 212 and the second combining portion 222 that match with one another.

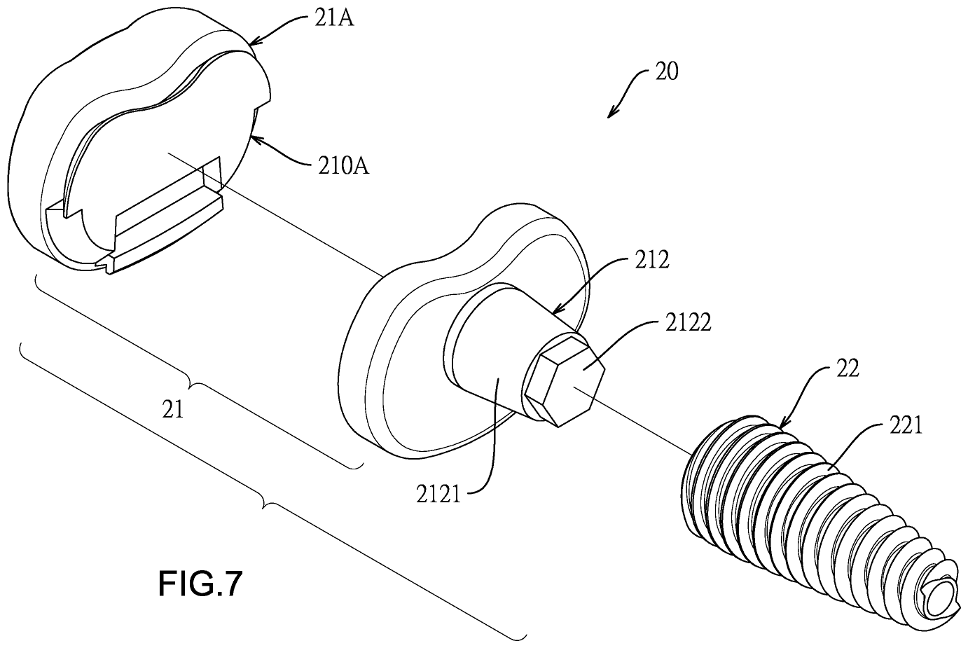

Specifically, with further reference to FIGS. 6 and 7, the second joint head element 21 has a second joint head portion 211. The second articular facet is formed on an end of the second joint head portion 211. The second connecting portion 212 is formed at another end of the second joint head portion 211. The second combining portion 222 is formed at an end of the second joint root element 22. The second connecting portion 212 and the second combining portion 222 is concave-convex matching structure.

The second connecting portion 212 and the second combining portion 222 of the second connecting structure are substantially the same as the first connecting portion 112 and the first combining portion 122 of the first connecting structure. Specifically, the second combining portion 222 has a conical recess 2221 and a polygonal recess 2222. The conical recess 2221 of the second combining portion 222 tapers off toward an inner bottom that is defined in said conical recess 2221. The polygonal recess 2222 of the second combining portion 222 is formed in the inner bottom that is defined in the conical recess 2221. The second connecting portion 212 has a conical protrusion 2121 and a polygonal protrusion 2122. The conical protrusion 2121 of the second connecting portion 212 tapers off from a proximal end of the conical protrusion 2121 to a distal end of the conical protrusion 2121. The polygonal protrusion 2122 of the second connecting portion 212 is formed on the distal end of the conical protrusion 2121 of the second connecting portion 212. The conical protrusion 2121 of the second connecting portion 212 is capable of being fitted in the conical recess 2221 of the second combining portion 222. The conical protrusion 2121 and the conical recess 2221 of the second combining portion 222 may be circular or polygonal in cross-section. The polygonal protrusion 2122 of the second connecting portion 212 is capable of being fitted in the polygonal recess 2222 of the second combining portion 222. With the mutually fitted conical protrusion and recess and the mutually fitted polygonal protrusion and recess, the second connecting portion 212 and the second combining portion 222 are able to be connected with each other quickly and stably.

As shown in FIG. 2, the second joint head element 21 may be an integrally formed single part, or as shown in FIGS. 3, 6 and 7, the second joint head element 21 may be formed by attaching an upper part 21A and a lower part 21B. The upper part 21A and the lower part 21B are connected with each other through a mortise-tenon structure. The second joint head portion 211 is formed on the upper part 21A. The second combining portion 212 is formed on the lower part 21B. The mortise-tenon structure includes a tenon 210A and a mortise 210B. The tenon 210A is formed on a side of the upper part 21A and is disposed opposite to the second connecting portion 211. The mortise 210B is formed on a side of the lower part 21B and is disposed opposite to the second connecting portion 212. The upper part 21A and the lower part 21B are stably connected with each other through the tenon 210A and the mortise 210B and are detachable from each other.

In an artificial joint replacement surgery, the first finger joint module 10 and the second finger joint module 20 of the modular artificial finger joint implant of the present invention are mounted to a first phalange and a second phalange of an injured finger or toe of the human body respectively and work as a finger joint or a toe joint such as a metacarpophalangeal joint, an interphalangeal joint, a proximal interphalangeal joint or the like.

The first joint root element 12 of the first finger joint module 10 is screwed to the first phalange and the second joint root element 22 of the second finger joint module 20 is screwed to the second phalange. With the thread 121 of the first joint root element 12 and the thread 221 of the second joint root element 22 and the sub-micron holes on the outer side surfaces of the first and second joint root elements 12, 22, in addition to be connected with the first phalange and the second phalange stably, the first joint root element 12 and the second joint root element 22 are also suitable for the attachment of the bone cells, which is beneficial for the integration of the first phalange and the first finger joint module 10 and the integration of the second phalange and the second finger joint module 20. Moreover, with the first joint head element 11 of the first finger joint module 10 and the second joint head element 21 of the second finger joint module 20 that are slidable relative to each other, the modular artificial finger joint implant of the present invention can work like a real finger joint.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

1. A modular artificial finger joint implant comprising:
a first finger joint module including
a first joint head element having a first articular facet;
a first joint root element; and
a first connecting structure disposed between and connecting the first joint head element and the first joint root element, and including
a first connecting portion; and
a first combining portion, wherein the first connecting portion and the first combining portion are detachably connected with each other; and
a second finger joint module including
a second joint head element having a second articular facet, and the second articular facet being slidable relative to the first articular facet of the first joint head element;
a second joint root element; and
a second connecting structure disposed between and connecting the second joint head element and the second joint root element, and including
a second connecting portion; and
a second combining portion, wherein the second connecting portion and the second combining portion are detachably connected with each other.

2. The modular artificial finger joint implant as claimed in claim 1, wherein the first joint root element and the second joint root element are conical-shaped.

3. The modular artificial finger joint implant as claimed in claim 1, wherein
the first joint root element is conical-shaped and has a thread formed on an outer side surface of the first joint root element; and
the second joint root element is conical-shaped and has a thread formed on an outer side surface of the second joint root element.

4. The modular artificial finger joint implant as claimed in claim 1, wherein
the first joint head element of the first connecting structure has a first joint head portion, the first articular facet is formed on an end of the first joint head portion, the first connecting portion is formed at another end of the first joint head portion, and the first combining portion is formed at an end of the first joint root element;
the second joint head element of the second connecting structure has a second joint head portion, the second articular facet is formed on an end of the second joint head portion, the second connecting portion is formed at another end of the second joint head portion, and the second combining portion is formed at an end of the second joint root element;
each of the first combining portion and the second combining portion has
a conical recess tapering off toward an inner bottom that is defined in said conical recess; and
a polygonal recess formed in the inner bottom that is defined in the conical recess; and
each of the first connecting portion and the second connecting portion has
a conical protrusion tapering off from a proximal end of the conical protrusion to a distal end of the conical protrusion and fitted in the conical recess of a corresponding one of the first combining portion and the second combining portion; and
a polygonal protrusion formed on the distal end of the conical protrusion and fitted in the polygonal recess of a corresponding one of the first combining portion and the second combining portion.

5. The modular artificial finger joint implant as claimed in claim 2, wherein
the first joint head element of the first connecting structure has a first joint head portion, the first articular facet is formed on an end of the first joint head portion, the first connecting portion is formed at another end of the first joint head portion, and the first combining portion is formed at an end of the first joint root element;
the second joint head element of the second connecting structure has a second joint head portion, the second articular facet is formed on an end of the second joint head portion, the second connecting portion is formed at another end of the second joint head portion, and the second combining portion is formed at an end of the second joint root element;
each of the first combining portion and the second combining portion has
a conical recess tapering off toward an inner bottom that is defined in said conical recess; and
a polygonal recess formed in the inner bottom that is defined in the conical recess; and
each of the first connecting portion and the second connecting portion has
a conical protrusion tapering off from a proximal end of the conical protrusion to a distal end of the conical protrusion and fitted in the conical recess of a corresponding one of the first combining portion and the second combining portion; and
a polygonal protrusion formed on the distal end of the conical protrusion and fitted in the polygonal recess of a corresponding one of the first combining portion and the second combining portion.

6. The modular artificial finger joint implant as claimed in claim 3, wherein
the first joint head element of the first connecting structure has a first joint head portion, the first articular facet is formed on an end of the first joint head portion, the first connecting portion is formed at another end of the first joint head portion, and the first combining portion is formed at an end of the first joint root element;
the second joint head element of the second connecting structure has a second joint head portion, the second articular facet is formed on an end of the second joint head portion, the second connecting portion is formed at another end of the second joint head portion, and the second combining portion is formed at an end of the second joint root element;
each of the first combining portion and the second combining portion has
a conical recess tapering off toward an inner bottom that is defined in said conical recess; and
a polygonal recess formed in the inner bottom that is defined in the conical recess; and
each of the first connecting portion and the second connecting portion has
a conical protrusion tapering off from a proximal end of the conical protrusion to a distal end of the conical protrusion and fitted in the conical recess of a corresponding one of the first combining portion and the second combining portion; and
a polygonal protrusion formed on the distal end of the conical protrusion and fitted in the polygonal recess of a corresponding one of the first combining portion and the second combining portion.

7. The modular artificial finger joint implant as claimed in claim 1, wherein the second joint head element is formed by attaching an upper part and a lower part, the second joint head portion is formed on the upper part, the second combining portion is formed on the lower part, and the upper part and the lower part are connected with each other through a mortise-tenon structure.

8. The modular artificial finger joint implant as claimed in claim 2, wherein the second joint head element is formed by attaching an upper part and a lower part, the second joint head portion is formed on the upper part, the second combining portion is formed on the lower part, and the upper part and the lower part are connected with each other through a mortise-tenon structure.

9. The modular artificial finger joint implant as claimed in claim 3, wherein the second joint head element is formed by attaching an upper part and a lower part, the second joint head portion is formed on the upper part, the second combining portion is formed on the lower part, and the upper part and the lower part are connected with each other through a mortise-tenon structure.

10. The modular artificial finger joint implant as claimed in claim 7, wherein
the mortise-tenon structure includes
a tenon formed on a side of the upper part and disposed opposite to the second connecting portion; and
a mortise formed on a side of the lower part and disposed opposite to the second connecting portion; and
the upper part and the lower part are stably connected with each other through the tenon and the mortise and are detachable from each other.

11. The modular artificial finger joint implant as claimed in claim 8, wherein
the mortise-tenon structure includes
a tenon formed on a side of the upper part and disposed opposite to the second connecting portion; and
a mortise formed on a side of the lower part and disposed opposite to the second connecting portion; and
the upper part and the lower part are stably connected with each other through the tenon and the mortise and are detachable from each other.

12. The modular artificial finger joint implant as claimed in claim 9, wherein
the mortise-tenon structure includes
a tenon formed on a side of the upper part and disposed opposite to the second connecting portion; and
a mortise formed on a side of the lower part and disposed opposite to the second connecting portion; and
the upper part and the lower part are stably connected with each other through the tenon and the mortise and are detachable from each other.
